(19) United States
(12) Patent Application Publication
(10) Pub. No.: US 2024/0059988 A1
(43) Pub. Date: Feb. 22, 2024

(54) PRODUCING OLEFINS AND AROMATICS

(71) Applicant: SABIC GLOBAL TECHNOLOGIES B.V., BERGEN OP ZOOM (NL)

(72) Inventors: Ravichander NARAYANASWAMY, Bangalore (IN); Alexander STANISLAUS, Bangalore (IN); Girish KORIPELLY, Bangalore (IN)

(73) Assignee: SABIC GLOBAL TECHNOLOGIES B.V., BERGEN OP ZOOM (NL)

(21) Appl. No.: 18/259,468

(22) PCT Filed: Nov. 18, 2021
(86) PCT No.: PCT/IB2021/060710

§ 371 Date: Jun. 27, 2023

Related U.S. Application Data
(60) Provisional application No. 63/130,958, filed on Dec. 28, 2020.

(51) Int. Cl. C10G 69/04; C07C 4/06

(52) U.S. Cl. CPC C10G 69/04; C07C 4/06; C10G 2400/20; C07C 2529/40; C10G 2400/30

(57) ABSTRACT

Systems and methods for processing hydrocarbons are disclosed. A crude oil, a crude oil fraction, and/or plastic pyrolysis oil is processed in a low pressure hydroprocessing unit to produce a cracker feed stream. The cracker feed stream is then flowed into a fluid catalytic cracking unit or a stream cracking unit to produce high value chemicals.

### CROSS REFERENCE TO RELATED APPLICATIONS

This application claims the benefit of priority to U.S. Provisional Patent Application No. 63/130,958, filed Dec. 28, 2020, the entire contents of which are hereby incorporated by reference in their entirety.

### FIELD OF INVENTION

The present invention generally relates to systems and methods for producing olefins and aromatics. More specially, the present invention relates to systems and methods for producing olefins and aromatics using hydroprocessed pyrolysis oil (pyoil) and/or at least a fraction of crude oil.

### BACKGROUND OF THE INVENTION

Cracking of naphtha is one of the most commonly used processes in the chemical industry for producing olefins and aromatics. In the last few decades, other fractions of crude oil, including atmospheric residue, vacuum gas oil, and naphtha/condensate have been used as feedstocks for cracking units. Other materials, including pyoil derived from plastics, have also been used as feedstock for cracking to produce olefins and aromatics.

Generally, cracking includes fluid catalytic cracking and steam cracking. Fluid catalytic cracking is a process commonly used in the chemical industry for producing olefins and aromatics. However, the yield of high severity chemicals like light gas olefins and aromatics is dependent on the hydrogen content of the feed, and for typical atmospheric residue or vacuum gas oil the hydrogen content is deficient and is around 12 to 12.5 wt. %, limiting the possibility to produce high yields of high value chemicals. A second problem is the higher coke formation from hydrogen deficient feed, which can limit the catalyst activity and reduce production of high value chemicals. Therefore, there is a need for upgrading these hydrogen deficient feeds in a way that can help get higher yields of high value chemicals when processed through a fluid catalytic cracking unit. For steam cracking of naphtha/condensate, the produced ethylene content is relatively lower for a higher boiling range feed as compared to a lower boiling range feed, resulting in lower economic benefits for the steam cracking process along with production of more fuel oils. In order to improve the economic benefit, it is thus desirable to process lower boiling range feed with potential to form more light gas olefins and less fuel oil.

Therefore, this drives the need for improvements in this field in light of at least the aforementioned drawbacks for the conventional systems and methods.

### BRIEF SUMMARY OF THE INVENTION

A solution to at least some of the above mentioned problems associated with systems and methods for cracking hydrocarbons is discovered. The solution resides in a method that includes hydroprocessing a pyrolysis oil obtained from a plastic and/or at least a fraction of crude oil. This can be beneficial for converting heavy fractions such as atmospheric residue and vacuum gas oil into saturated crude oil fractions (e.g., saturated atmospheric residue and saturated vacuum gas oil), thereby minimizing issues caused by coking of feedstocks for cracking processes. Additionally, plastic derived pyoil can be produced and fed into a hydroprocessing unit to saturate the pyoil and to remove chlorine from the pyoil, as well as other heteroatom contaminants. The hydroprocessed pyoil can be used as part of the feedstock for the cracking process, resulting in higher reuse value for plastics. Furthermore, the hydroprocessing can be conducted in a hydroprocessing unit (e.g., a fixed bed hydroprocessing unit, ebullated bed hydroprocessing unit, or a slurry hydroprocessing unit) that operates at less than 100 barg and intensifies hydroprocessing by using a combination of dissolved and fixed bed catalysts to provide thorough hydrogenation as well as access through molecular catalysts to less accessible sites, resulting in higher hydroprocessing conversions. Moreover, the hydroprocessing unit for hydroprocessing can be operated in a hydrocracking mode and/or hydrotreating mode and optimize the consumption of hydrogen by removing an amount of carbon from the process as coke. Therefore, the methods of the present invention provide a technical solution to at least some of the problems associated with the conventional methods and systems for cracking hydrocarbons.

Embodiments of the invention include a method of processing hydrocarbons. The method comprises hydroprocessing, in a hydroprocessing unit, a hydrocarbon stream comprising (1) a pyrolysis oil obtained from a plastic and (2) at least a fraction from a crude oil distillation unit under reaction conditions sufficient to produce a cracker feed stream. The method comprises cracking, in a fluid catalytic cracking unit, hydrocarbons of the cracker feed stream in the presence of a catalyst under reaction conditions sufficient to produce a product stream comprising one or more of C₂ olefins, C₃ olefins, C₄ olefins, benzene, toluene, and xylenes.

Embodiments of the invention include a method of processing hydrocarbons. The method comprises hydroprocessing, in a hydroprocessing unit, a liquid fraction from a crude oil distillation unit under reaction conditions sufficient to produce a hydroprocessed liquid stream. The method comprises mixing the hydroprocessed liquid stream and a pyrolysis oil obtained from a plastic to form a cracker feed stream. The method comprises cracking, in a fluid catalytic cracking unit, hydrocarbons of the cracker feed stream in the presence of a catalyst under reaction conditions sufficient to produce a product stream comprising one or more of C₂ olefins, C₃ olefins, C₄ olefins, benzene, toluene, and xylene.

Embodiments of the invention include a method of processing hydrocarbons. The method comprises processing a plastic stream comprising a thermally cracked and/or partially thermally cracked plastic in a fixed bed or fluidized bed catalytic cracking unit under reaction conditions sufficient to crack at least some hydrocarbons of the thermally cracked and/or partially thermally cracked plastic, removing at least some of chlorine from the thermally cracked and/or partially thermally cracked plastic, and producing a pyrolysis oil stream comprising pyrolysis oil. The method includes hydroprocessing, in a hydroprocessing unit, the pyrolysis oil stream and crude oil or a crude oil fraction to produce a cracker feed stream. The method includes cracking hydrocarbons of the cracker feed stream in a cracking unit under reaction conditions sufficient to produce a product stream comprising one or more C₂ olefins, C₃ olefins, C₄ olefins, benzene, toluene, and xylene.

Embodiments of the invention include a method of processing hydrocarbons. The method comprises processing a plastic stream comprising a thermally cracked and/or partially thermally cracked plastic in a fixed bed or fluid catalytic cracking unit under reaction conditions sufficient to crack at least some hydrocarbons of the thermally cracked and/or partially thermally cracked plastic, remove at least some of chlorine from the thermally cracked and/or partially thermally cracked plastic, and produce a pyrolysis oil stream comprising pyrolysis oil. The method comprises hydroprocessing, in a hydroprocessing unit, the pyrolysis oil stream to produce a hydroprocessed pyrolysis oil stream. The method comprises mixing the hydroprocessed pyrolysis oil stream with crude oil or a crude oil fraction to produce a cracker feed stream. The method comprises cracking hydrocarbons of the cracker feed stream in a cracking unit under reaction conditions sufficient to produce a product stream comprising one or more of C₂ olefins, C₃ olefins, C₄ olefins, benzene, toluene, and xylene.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1A shows a schematic diagram of a first system for processing a hydrocarbon feed in a fluid catalytic cracking unit, according to embodiments of the invention;

FIG. 1B shows a schematic diagram of a second system for processing a hydrocarbon feed in a fluid catalytic cracking unit, according to embodiments of the invention;

FIG. 1C shows a schematic diagram of a third system for processing a hydrocarbon feed in a fluid catalytic cracking unit, according to embodiments of the invention;

FIG. 2A shows a schematic diagram of a first system for processing a hydrocarbon feed in a steam cracker, according to embodiments of the invention;

FIG. 2B shows a schematic diagram of a second system for processing a hydrocarbon feed in a steam cracker, according to embodiments of the invention;

FIG. 2C shows a schematic diagram of a third system for processing a hydrocarbon feed in a steam cracker, according to embodiments of the invention;

FIG. 3A shows a schematic flowchart of a method of processing a hydrocarbon feed that can be implemented in the systems shown in FIG. 1A and/or FIG. 1B, according to embodiments of the invention;

FIG. 3B shows a schematic flowchart of a method of processing a hydrocarbon feed that can be implemented in the systems shown in FIG. 1C, according to embodiments of the invention;

FIG. 3C shows a schematic flowchart of a method of processing a hydrocarbon feed that can be implemented in the systems shown in FIG. 2A and/or FIG. 2B, according to embodiments of the invention; and

FIG. 3D shows a schematic flowchart of a method of processing a hydrocarbon feed that can be implemented in the systems shown in FIG. 2C, according to embodiments of the invention.

### DETAILED DESCRIPTION OF THE INVENTION

Currently, hydrocarbon cracking technology suffers several drawbacks. Yield of high severity chemicals like light gas olefins and aromatics is generally dependent on the hydrogen content of the feed and for typical atmospheric residue or vacuum gas oil, and the hydrogen content generally around 12 to 12.5 wt. %, which is deficient, resulting in limited possibility to produce high yields of high value chemicals. A second problem is the higher coke formation from hydrogen deficient feed which would limit the catalyst activity and reduce production of high value chemicals. Therefore, there is need for upgrading of these hydrogen deficient feeds in a way that would help get higher yields of high value chemicals when processed through a fluid catalytic cracking unit. For steam cracking of naphtha/condensate, the produced ethylene content is relatively lower for a higher boiling range feed as compared to a lower boiling range feed, resulting in lower economic benefits for the steam cracking process, along with production of more fuel oils. In order to improve the economic benefit, it is thus desirable to process lower boiling range feed with potential to form more light gas olefins and less fuel oil. The present invention provides a solution to at least some of these problems. The solution is premised on a method of hydroprocessing a pyrolysis oil obtained from a plastic and/or at least a fraction of crude oil. The method is configured to minimize issues caused by coking of feedstocks for cracking processes by converting heavy fractions such as atmospheric residue and vacuum gas oil into saturated lighter crude oil fractions (e.g., saturated atmospheric distillates predominantly at less than 350° C.). Additionally, plastic derived pyoil can be processed in a hydroprocessing unit to saturate the pyoil and to remove chlorine from the pyoil, as well as cracking the pyoil to lower molecular weight liquid components. The hydroprocessed pyoil can be used as part of feedstock for the cracking process, increasing values for waste plastics. Furthermore, the hydroprocessing can be conducted in a hydroprocessing unit (e.g., a fixed bed hydroprocessing unit, ebullated bed hydroprocessing unit, or a slurry hydroprocessing unit). The hydroprocessing unit is configured to operate at less than 100 barg and intensify hydroprocessing by using a combination of dissolved and a conventional hydroprocessing catalyst, e.g., a fixed bed catalyst to provide thorough hydrogenation as well as access through molecular catalyst to less accessible sites, resulting in higher hydroprocessing conversion rates. Moreover, the hydroprocessing unit for hydroprocessing can be operated in a hydrocracking mode and/or hydrotreating mode and optimize the consumption of hydrogen by removing an amount of carbon as coke. These and other non-limiting aspects of the present invention are discussed in further detail in the following sections.

### A. Systems for Processing Hydrocarbons

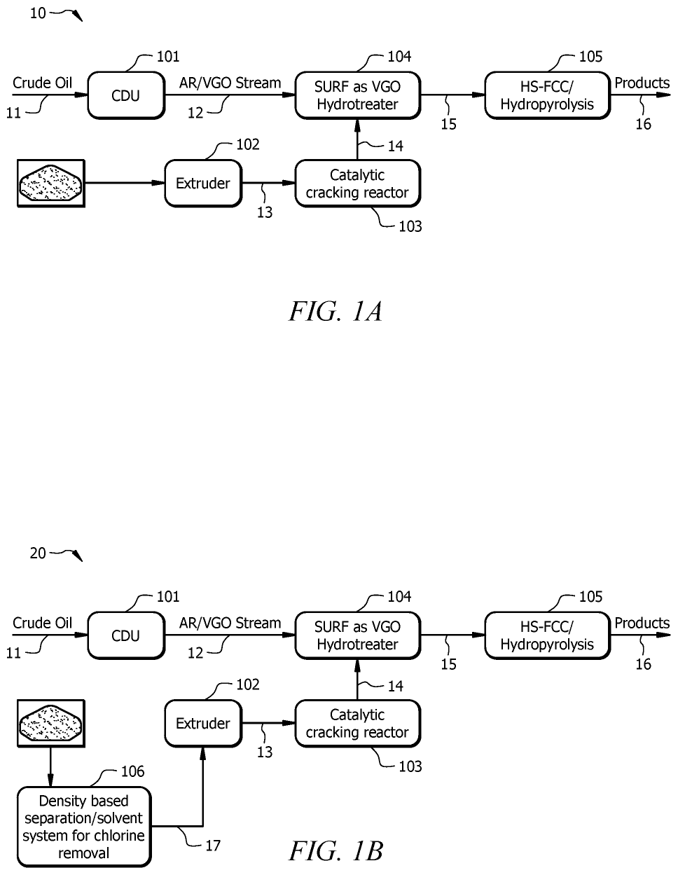

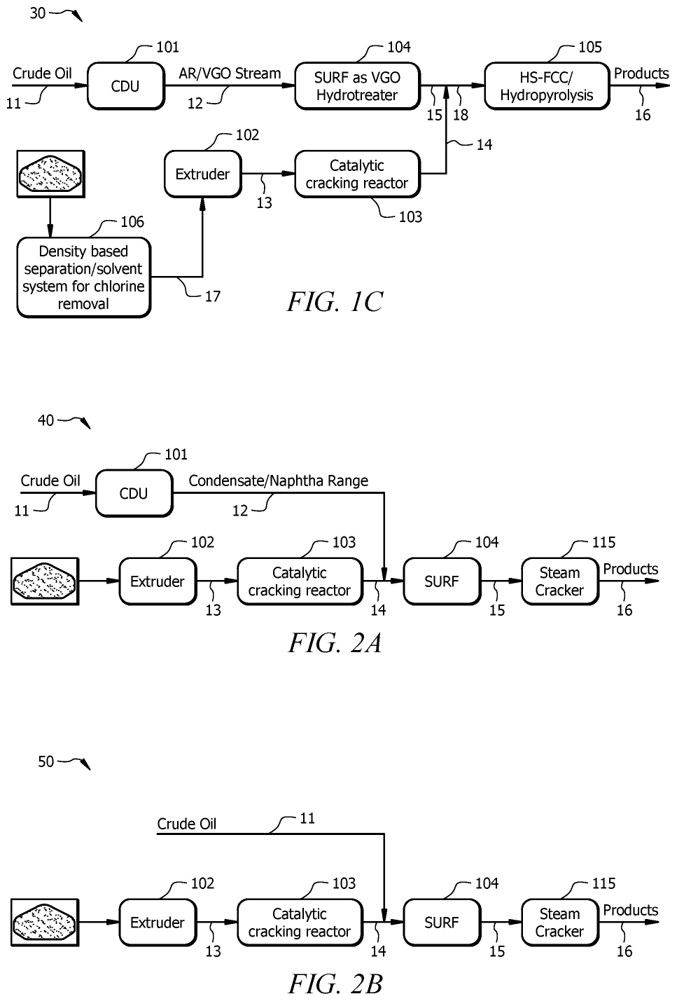

In embodiments of the invention, a system for processing hydrocarbons can include a hydroprocessing unit and a cracking unit. With reference to FIG. 1A, a schematic diagram is shown of system 10 for processing hydrocarbons.

According to embodiments of the invention, system 10 comprises distillation unit 101 (e.g., crude oil distillation unit (CDU)) configured to distill crude oil of crude oil stream 11 to produce first feedstock stream 12 comprising a fraction of crude oil. In embodiments of the invention, first feedstock stream 12 comprises atmospheric residue and/or vacuum gas oil (AR/VGO). In embodiments of the invention, distillation unit 101 comprises an atmospheric distillation column.

According to embodiments of the invention, system 10 includes extruder 102 configured to process plastic to produce first intermediate plastic stream 13 comprising hydrocarbonaceous wax stream having molecular weight of 5000 to 10000. In embodiments of the invention, one or more depolymerization additives are optionally mixed with the plastic in extruder 102. The depolymerization additives can include a depolymerization accelerator/organometallic compound, a cracking catalyst, or combinations thereof. The depolymerization accelerator/organometallic compound includes a metal octonoate, metal naphthenate, metal stearate, metallocenes, or combinations thereof. The metal in the organometallic compound includes Ni, Mo, Co, W, transitional metals, or combinations thereof. The solid catalyst/additives can be configured to accelerate the depolymerization rate in the extruder so that the targeted molecular weight reduction can be achieved at a reduced residence time. Exemplary solid catalysts/additives may include an inorganic oxide, aluminosilicates including ZSM-5, an X-type zeolite, a Y-type zeolite, a USY-zeolite, mordenite, faujasite, nano-crystalline zeolites, MCM mesoporous materials, SBA-15, a silico-alumino phosphate, a gallium phosphate, and a titanophosphate, a molecular sieve, and combinations thereof. In embodiments of the invention, the liquid catalyst/additive and/or the solid catalyst/additive are configured to scavenge chlorides and enhance production of straight chain hydrocarbons over branched hydrocarbons. Metal loaded aluminosilicates help in scavenging chlorides as well as enhancing straight chain hydrocarbons over branched hydrocarbons. In embodiments of the invention, extruder 102 includes extruder/auger/twin screw reactor, a piston in a feed chamber, a block and feed type of manifold, or combinations thereof. In embodiments of the invention, extruder 102 is a continuous feeding device. According to embodiments of the invention, this feeding device is equipped with an automated valving system. The automated valving system may be configured for gas pressurized discharge being operated in a timer-based pulse mode for mimicking continuous feeding. A continuous screw feeder is used for metering the flow of plastics to the solid hopper of the extruder. The valve below the solid hopper will be kept on during operation and will be closed only for maintenance. The idea of keeping the metering feeder includes the solid hopper does not accumulate solids and the throughput of the extruder is controlled by the metering feeder for different residence time in the extruder. Instead of a screw feeder, a pressurized hopper with a rotary valving arrangement set to operate on a timer-based operation (to mimic continuous flow) can also be used to feed extruder 102. The timer of the rotary valve can be adjusted for different feeding rates to the extruder for different residence times.

A third option is that the pressurized hopper outlet has two timer operation-based solenoid valves in series connected by means of a pipe to feed the solid hopper feeding the extruder. The solenoid valves are operated such that at any given point in time one solenoid valve is open, e.g., if the solenoid valve directly below the hopper is in an open condition, then the next solenoid valve is in a closed condition to allow filling of the pipe between the solenoid valves. After a certain time, the first valve closes and the second valve opens to feed the plastic to the solid hopper feeding the extruder. The frequency of opening and closing these solenoid valves will determine the feeding rate and thus the residence time in the extruder. This is an example of block and manifold type automated valving. The extruder can have a single screw, left-handed screw, right-handed screw, neutral screw, kneading screw, multiple screws, intermeshing co-rotating or counter-rotating screws, non-intermeshing, co-rotating, or counter-rotating screws, reciprocating screws, screws with pins, screws with screens, barrels with pins, rolls, rams, helical rotors, co-kneaders, disc-pack processors, various other types of extrusion equipment, or combinations comprising at least one of the foregoing. The plastics feed in the extruder barrel can be heated with one or more heaters arranged along the length of the extruder barrel. In the extruder barrel, the plastic feed can be heated, melted, and depolymerized to form the hydrocarbonaceous wax. In some aspects, the plastics feed in the extruder barrel can be depolymerized at a temperature of 300° C. to 500° C., or at least any one of, equal to one of, or between any two of 300, 320, 340, 360, 380, 400, 420, 440, 450, 480, and 500° C. The residence time of the plastics in the extruder can be less than an hour, such as 1 min. to 15 min. In certain aspects, the extruder can contain one or more vents configured to introduce and/or withdraw one or more gases into and/or from the extruder barrel. The plastic melt and/or hydrocarbonaceous wax can be extruded from the extruder through a die.

According to embodiments of the invention, an outlet of extruder 102 is in fluid communication with an inlet of catalytic cracking unit 103 such that first intermediate plastic stream 13 flows from extruder to catalytic cracking unit 103. In embodiments of the invention catalytic cracking unit 103 is configured to crack first intermediate plastic stream 13 to produce pyoil stream 14 comprising paraffins, isoparaffins, olefins, naphthenes, and aromatics. In embodiments of the invention, catalytic cracking unit 103 includes a fixed bed reactor, a fluidized bed reactor, a stirred tank reactor, rotary kilns, preferably a fixed bed reactor and a continuous stirred tank reactor. In embodiments of the invention, catalytic cracking unit 103 includes a catalyst comprising ZSM-5, metal loaded ZSM-5, spent fluid catalytic cracking catalyst, or any combinations thereof.

According to embodiments of the invention, an outlet of distillation unit 101 is in fluid communication with an inlet of hydroprocessing unit 104 such that first feedstock stream 12 flows from distillation unit 101 to hydroprocessing unit 104. In embodiments of the invention, an outlet of catalytic cracking unit 103 is in fluid communication with an inlet of hydroprocessing unit 104 such that pyoil stream 14 flows from catalytic cracking unit 103 to hydroprocessing unit 104. Hydroprocessing unit 104 is configured to hydrotreat and/or hydrocrack hydrocarbons of first feedstock stream 12 and/or pyoil stream 14 to produce cracker feed stream 15 comprising hydroprocessed fraction of the crude oil and hydroprocessed pyoil. In embodiments of the invention, hydroprocessing includes removal at least some chlorine from pyoil stream 14. In embodiments of the invention, hydroprocessing unit 104 can include a fixed bed hydroprocessing unit, ebullated bed hydroprocessing unit, or a slurry hydroprocessing unit. Hydroprocessing unit 104 can have disposed therein hydrotreating catalysts and/or hydrocracking catalysts. Exemplary hydrotreating catalysts can include CoMo, NiMo, CoNiMo, NiW, NiWMo, and combinations thereof on alumina or silica or aluminosilicates. Exemplary hydrocracking catalysts can include CoMo, NiMo, CoNiMo, NiW, NiWMo, or combinations thereof on alumina, silica, aluminosilicates or zeolites such as X-type zeolites, Y-type or USY-type zeolites, mordenite, faujasite, nano-crystalline zeolites, MCM mesoporous materials, SBA-15, silico-alumino phosphate, gallophosphate, titanophosphate, ZSM-5, ZSM-11, ferrierite, heulandite, zeolite-A, erionite, and chabazite, and combinations thereof.

In embodiments of the invention, hydroprocessing unit 104 (e.g., a fixed bed hydroprocessing unit, ebullated bed hydroprocessing unit, or a slurry hydroprocessing unit) is configured to operate at a pressure of less than 100 barg. Hydroprocessing unit 104 can be configured to intensify hydroprocessing process by using a combination of dissolved and solid (fixed bed) catalyst to provide deep hydrogenation and enabling access through molecular catalyst to diffuse to access sites for higher hydroprocessing conversion rate. According to embodiments of the invention, hydroprocessing unit 104 is configured to be able to operate in a hydrocracking mode and/or a hydrotreating mode. Hydroprocessing unit 104 can be configured to optimize hydrogen consumption via rejection of carbon as coke.

In embodiments of the invention, for system 10, an outlet of hydroprocessing unit 104 is in fluid communication with an inlet of fluid catalytic cracking unit 105 such that cracker feed stream 15 flows from hydroprocessing unit 104 to fluid catalytic cracking unit 105. In embodiments of the invention, fluid catalytic cracking unit 105 is configured to crack hydrocarbons of cracker feed stream 15 to produce product stream 16. Product stream 16 comprises high valued chemicals including olefins, benzene, toluene, xylene (BTX).

According to embodiments of invention, as shown in FIG. 1B, system 20 includes all the units and stream of system 10 (as described above). In embodiments of the invention, system 20 further includes chlorine removal unit 106 configured to remove chlorine from plastic to produce low chlorine plastic stream 17. Low chlorine plastic stream 17 comprises 1 to 1000 ppm chlorine. In embodiments of the invention, chlorine removal unit 106 comprises a density based separation and/or solvent system configured to remove chlorine from plastic. Chlorine removal unit 106 can be configured to remove heteroatom containing polymer flakes from the plastic. Exemplary heteroatom containing polymer flakes can include polyvinyl chloride (PVC) and polyvinylidene dichloride (PVDC). An outlet of chlorine removal unit 106 is in fluid communication with an inlet of extruder 102 such that low chlorine plastic stream 17 flows from chlorine removal unit 106 to extruder 102. In embodiments of the invention, feedstock stream 12 along with pyoil stream 20 forms a feed mixture to hydroprocessing unit 105, and cracker feed stream 15 is fed to fluid catalytic cracking unit 105 configured to produce product stream 16.

According to embodiments of invention, as shown in FIG. 1C, system 30 includes all the units and stream of system 20 (as described above) except that at least some of pyoil stream 14 is combined with cracker feed stream 15 to form combined cracker feed stream 18. Combined cracker feed stream 18 can be flowed into fluid catalytic cracking unit 105.

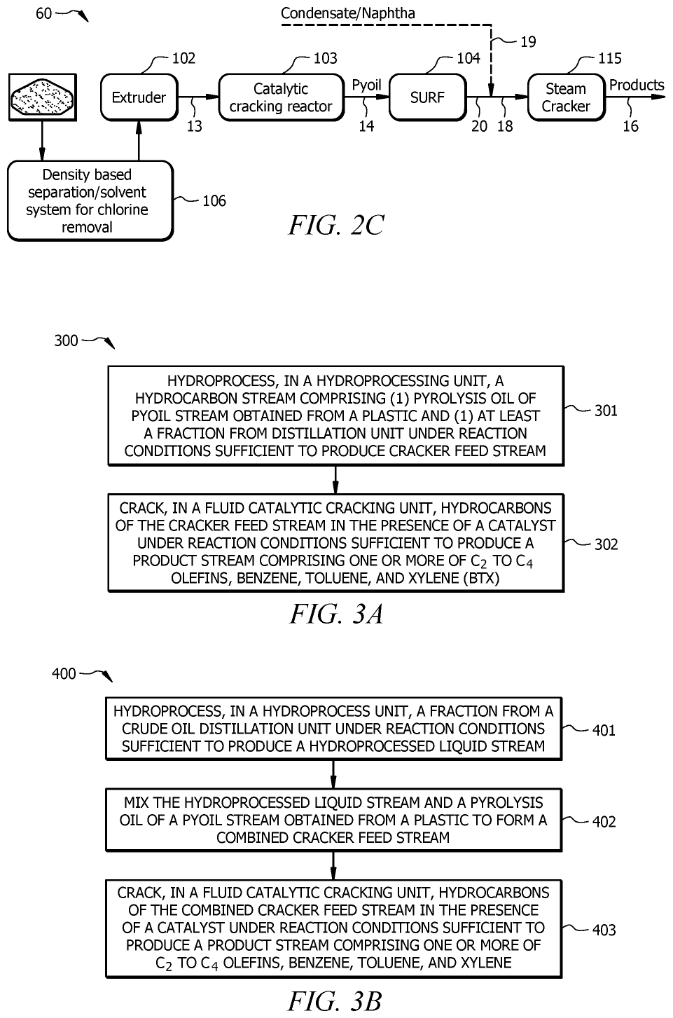

According to embodiments of the invention, as shown in FIG. 2A, system 40 includes all the streams and units as system 10 (as described above) except first feedstock stream 12 includes condensate and/or naphtha boiling range stream. System 40 includes steam cracker 115 in place of fluid catalytic cracking unit 105.

According to embodiments of the invention, as shown in FIG. 2B, system 50 includes all the streams and units as system 40 (as described above) except that system 50 does not include crude distillation unit 101 such that crude oil stream 11 can be mixed with pyoil stream 14 and fed into hydroprocessing unit 104. In embodiments of the invention, in system 50, crude oil stream 11 includes light crude oil that has an initial boiling point of less than 35 to 35° C. and a final boiling point of 350 to 500° C.

According to embodiments of the invention, as shown in FIG. 2C, system 60 includes all the streams and units of system 40 except that system 60 does not include distillation unit 101. Second fraction stream 19 of system 40 including condensate and/or naphtha can be combined with cracker feed stream 15 and fed into steam cracker 115, and plastic is first processed in chlorine removal unit 106. An outlet of chlorine removal unit 106 is in fluid communication with an inlet of extruder 102 such that low chlorine plastic stream 17 flows from chlorine removal unit 106 to extruder 102.

### B. Methods of Processing Hydrocarbons

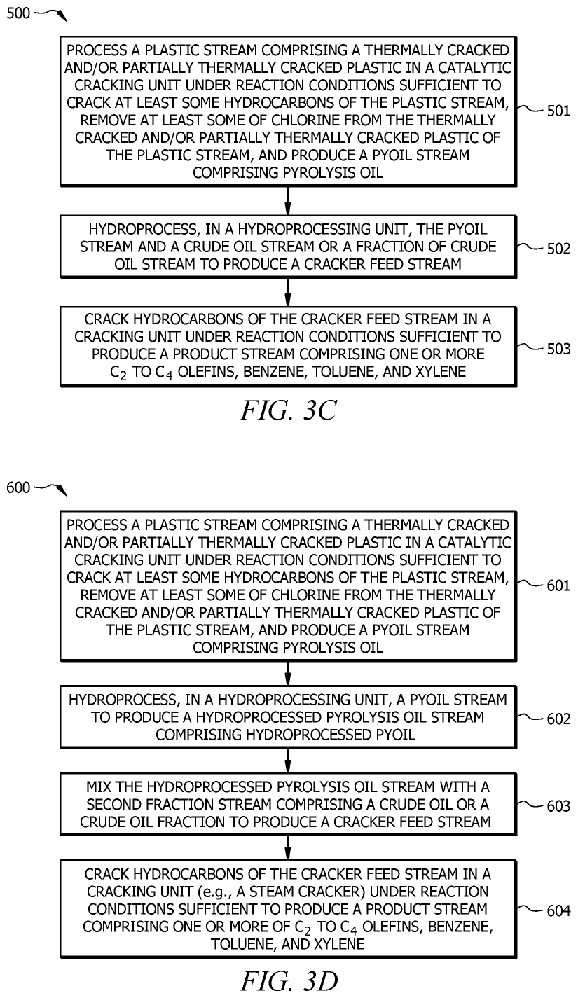

Methods of processing hydrocarbons have been discovered. The methods are capable of improving the cracking efficiency via mitigating coking of feedstock and/or adding hydrogen to heavy feedstocks including condensate and/or naphtha. As shown in FIG. 3A, embodiments of the invention include method 300 for processing hydrocarbons. Method 300 can be implemented by system 10 and/or system 20, as shown in FIG. 1A and FIG. 1B, respectively.

According to embodiments of the invention, as shown in block 301, method 300 includes hydroprocessing, in hydroprocessing unit 104, a hydrocarbon stream comprising (1) pyrolysis oil of pyoil stream 14 obtained from a plastic and (2) at least a fraction (first feedstock stream 12) from distillation unit 101 or (3) whole crude oil (crude oil stream 11) under reaction conditions sufficient to produce cracker feed stream 15. In embodiments of the invention, first feedstock stream 12 includes atmospheric residue and/or vacuum gas oil. First feedstock stream 12 may be obtained by distilling crude oil stream 11 in distillation unit 101. The feedstock can also include crude oil stream 11. In embodiments of the invention, the hydroprocessing at block 301 comprises low pressure hydrocracking/hydrotreating. The low pressure hydrocracking and/or hydrotreating is configured to produce cracker feed stream 15 having a boiling range of light vacuum gasoil with a boiling range of 350 to 475° C. The low pressure hydrocracking and/or hydrotreating is configured to further produce an intermediate stream, and/or a liquefied petroleum gas (LPG) stream. The intermediate stream and/or the LPG can be processed in a cracking unit to produce one or more olefins and/or one or more aromatics (e.g., benzene, toluene, and xylene). The intermediate stream may have a boiling range of less than 35° C. to 350° C. At block 301, hydroprocessing unit 104 may be operated as a hydrotreater for vacuum gas oil. Hydroprocessing unit 104, at block 301 is operated to provide a hydroprocessing temperature of 300 to 400° C. and a hydroprocessing pressure of 20 to 60 barg. In embodiments of the invention, at block 301, hydroprocessing unit 104 is operated with a weight hourly space velocity of 1 to 2 hr⁻¹ and all ranges and values there between. Hydroprocessing unit 104 may be operated to provide a hydrogen to hydrocarbon ratio of 400 to 2000 Nm³/m³ liquid feed (normal cubic meter). In embodiments of the invention, hydroprocessing unit 104 can further remove heteroatoms including chlorine and produce a feedstock to the FCC unit (cracker feed stream 15), which is rich in hydrogen (>12.5 wt. %) and has good crackability and low coking tendency. This feedstock of cracker feed stream 15 can have a boiling point distribution with an initial boiling point range of less than 35 to 35° C. and a final boiling point range of 350° C. to 500° C.

In embodiments of the invention, pyrolysis oil of pyoil stream 14 is derived from processing the plastic in extruder 102 to produce first intermediate plastic stream 13 and cracking first intermediate plastic stream 13 in catalytic cracking unit 103 to produce pyoil stream 14. In embodiments of the invention, prior to processing in extruder 102, the plastic may be processed in chlorine removal unit 106 to remove at least some chlorine prior to being processed in extruder 102. Chlorine removal unit 106 may be configured to remove more than 90% chlorine from the plastic. In embodiments of the invention, the plastic includes polyolefins, polystyrene, polyethylene terephthalate (PET), polyvinyl chloride (PVC), polyvinylidene chloride (PVDC), polyamides, or combinations thereof. In embodiments of the invention, extruder 102 is operated to provide a temperature of 300 to 450° C. Catalytic cracking unit 103 is operated to provide a reaction temperature of 350 to 500° C. and a pressure of 1 to 6 bara and a residence time of 1 hr or less. Pyoil stream 14 may have a boiling range of less than 35 to 750° C.

According to embodiments of the invention, as shown in block 302, method 300 includes cracking in fluid catalytic cracking unit 105, hydrocarbons of cracker feed stream 15 in the presence of the catalyst under reaction conditions sufficient to produce product stream 16 comprising one or more of C₂ to C₄ olefins, benzene, toluene, and xylene (BTX). In embodiments of the invention, product stream 16 can include 10 to 15 wt. % C₂ olefins, 15 to 35 wt. % C₃ olefins, 10 to 20 wt. % C₄ olefins, and 10 to 30 wt. % BTX. In embodiments of the invention, the reaction conditions at block 302 includes a reaction temperature of 450 to 750° C. The reaction conditions at block 302 may include a reaction pressure of 1 to 6 bara. In embodiments of the invention, fluid catalytic cracking unit 105 can additionally be operated under hydropyrolysis conditions in the presence of hydrogen or hydrogen-containing gas.

As shown in FIG. 3B, embodiments of the invention include method 400 for processing hydrocarbons. Method 400 can be implemented by system 30, as shown in FIG. 1C. According to embodiments of the invention, as shown in block 401, method 400 includes hydroprocessing, in hydroprocessing unit 104, a fraction (first feedstock stream 12) from distillation unit 101 or crude oil (crude oil stream 11) under reaction conditions sufficient to produce a hydroprocessed liquid stream (cracker feed stream 15). Cracker feed stream 15 may comprise less than 3 ppmw chlorine. In embodiments of the invention, first feedstock stream 12 includes atmospheric residue and/or vacuum gas oil. First feedstock stream 12 may be obtained by distilling crude oil stream 11 in distillation unit 101. In embodiments of the invention, the hydroprocessing at block 401 comprises low pressure hydrocracking and/or hydrotreating. At block 401, hydroprocessing unit 104 may be operated as a hydrotreater for vacuum gas oil. Hydroprocessing unit 104, at block 401 is operated to provide a hydroprocessing temperature of 300 to 600° C., preferably between 350 and 500° C., and a hydroprocessing pressure of 20 to 100 barg, according to embodiments of the invention. In embodiments of the invention, at block 401, hydroprocessing unit 104 is operated with a weight hourly space velocity of 1 to 2 hr⁻¹ and all ranges and values there between. Hydroprocessing unit 104 may be operated to provide a hydrogen to hydrocarbon ratio of 400 to 2000 Nm³/m³ liquid stream.

According to embodiments of the invention, as shown in block 402, method 400 includes mixing the hydroprocessed liquid stream (cracker feed stream 15) and a pyrolysis oil of pyoil stream 14 obtained from a plastic to form combined cracker feed stream 18. In embodiments of the invention, pyrolysis oil of pyoil stream 14 is derived from processing the plastic in extruder 102 to produce first intermediate plastic stream 13 and cracking first intermediate plastic stream 13 in catalytic cracking unit 103 to produce pyoil stream 14. In embodiments of the invention, the plastic may be processed in chlorine removal unit 106 to remove at least some chlorine prior to being processed in extruder 102. Chlorine removal unit 106 may be configured to remove more than 90% chlorine from the plastic. In embodiments of the invention, the plastic includes polyolefins, polystyrene, PET, PVC, PVDC, polyamides, or combinations thereof. In embodiments of the invention, extruder 102 is operated to provide a temperature of 300 to 450° C. Catalytic cracking unit 103 is operated to provide a reaction temperature of 350 to 500° C. and a pressure of 1 to 6 bara, in embodiments of the invention. Pyoil stream 14 may have a boiling range of less than 35 to 750° C. In embodiments of the invention, pyoil stream 14 includes less than 100 ppm preferably less than 30 ppm chlorine. Pyoil stream 14 may comprise less than 10 wt. % aromatics.

According to embodiments of the invention, as shown in block 403, method 400 includes cracking, in fluid catalytic cracking unit 105, hydrocarbons of combined cracker feed stream 18 in the presence of the catalyst of cracking unit 105 under reaction conditions sufficient to produce product stream 16 comprising one or more of C₂ to C₄ olefins, benzene, toluene, and xylene. In embodiments of the invention, product stream 16 can include 10 to 20 wt. % C₂ olefins, 10 to 30 wt. % C₃ olefins, 5 to 15 wt. % C₄ olefins, and 10 to 20 wt. % BTX. In embodiments of the invention, the reaction conditions at block 403 include a reaction temperature of 450 to 750° C. The reaction conditions at block 403 may include a reaction pressure of 1 to 6 bara. In embodiments of the invention, fluid catalytic cracking unit 105 can additionally be operated under hydropyrolysis conditions in the presence of hydrogen or hydrogen containing gas.

As shown in FIG. 3C, embodiments of the invention include method 500 for processing hydrocarbons. Method 500 can be implemented by systems 40 and/or 50, as shown in FIG. 2A and/or 2B, respectively. According to embodiments of the invention, as shown in block 501, method 500 includes processing plastic stream 13 comprising a thermally cracked and/or partially thermally cracked plastic in catalytic cracking unit 103 under reaction conditions sufficient to crack at least some hydrocarbons of plastic stream 13, remove at least some of chlorine from the thermally cracked and/or partially thermally cracked plastic of plastic stream 13, and produce pyoil stream 14 comprising pyrolysis oil. In embodiments of the invention, plastic stream 13 comprising thermally cracked and/or partially thermally cracked plastic is produced by processing the plastic in extruder 102. The extruder can be operated to provide an operating temperature of 300 to 450° C. and a residence time of 1 to 15 minutes.

In embodiments of the invention, at block 501, catalytic cracking unit 103 includes a fixed bed reactor, a fluidized bed reactor, a stirred tank reactor, rotary kilns, preferably a fixed bed reactor and a continuous stirred tank reactor. In embodiments of the invention, catalytic cracking unit 103 includes a catalyst comprising ZSM-5, metal loaded ZSM-5, spent fluid catalytic cracking catalyst, or any combinations thereof. The fixed bed catalytic cracking reactor can have ZSM-5 disposed in it as the catalyst. At block 501, catalytic cracking unit 103 can be operated to provide an operating temperature of 300 to 500° C., an operating pressure of 1 to 6 bara, and a residence time of 1 hr or less.

According to embodiments of the invention, as shown in block 502, method 500 includes hydroprocessing, in hydroprocessing unit 104, pyoil stream 14 and crude oil stream 11 or a fraction (i.e., first feedstock stream 12) of crude oil stream 11 to produce cracker feed stream 15. In embodiments of the invention, hydroprocessing unit 104 is operated to provide a reaction temperature of 350 to 500° C. and all ranges and values there between including ranges of 350 to 365° C., 365 to 380° C., 380 to 395° C., 395 to 410° C., 410 to 425° C., 425 to 440° C., 440 to 455° C., 455 to 470° C., 470 to 485° C., and 485 to 500° C. Hydroprocessing unit 104, at block 502, can be operated to provide an operating pressure of 20 to 100 barg and all ranges and values there between including ranges of 20 to 30 barg, 30 to 40 barg, 40 to 50 barg, 50 to 60 barg, 60 to 70 barg, 70 to 80 barg, 80 to 90 barg, and 90 to 100 barg. At block 502, hydroprocessing unit 104 can be operated to provide a weight hourly space velocity of 1 to 2 hr⁻¹. In embodiments of the invention, at block 502, hydroprocessing unit 104 can be operated to provide a hydrogen to hydrocarbon volumetric ratio of 400 to 2000 Nm³/m³ liquid feed. At block 502, cracker feed stream 15 comprises naphtha that has a boiling range below 150° C. In embodiments of the invention, at block 502, the processing conditions are adjusted such that cracker feed stream 15 comprises less than 3 ppm, preferably 1 ppm chlorine and less than 1 wt. % olefins.

According to embodiments of the invention, as shown in block 503, method 500 includes cracking hydrocarbons of cracker feed stream 15 in a cracking unit (e.g., steam cracker 115) under reaction conditions sufficient to produce product stream 16 comprising one or more C₂ to C₄ olefins, benzene, toluene, and xylene. In embodiments of the invention, product stream 16 can include 20 to 38 wt. % C₂ olefins, 10 to 20 wt. % C₃ olefin, 5 to 10 wt. % C₄ olefins, and 8 to 15 wt. % BTX. In embodiments of the invention, the reaction conditions at block 503 include a reaction temperature of 750 to 900° C. The reaction conditions at block 503 may include a reaction pressure of atmospheric pressure to 6 barg. In embodiments of the invention, steam cracking unit is operated with a residence time of 50 ms to 1 s.

As shown in FIG. 3D, embodiments of the invention include method 600 for processing hydrocarbons. Method 600 can be implemented by system 60, as shown in FIG. 2C. According to embodiments of the invention, as shown in block 601, method 600 includes processing plastic stream 13 comprising a thermally cracked and/or partially thermally cracked plastic in catalytic cracking unit 103 under reaction conditions sufficient to crack at least some hydrocarbons of plastic stream 13, remove at least some of chlorine from the thermally cracked and/or partially thermally cracked plastic of plastic stream 13, and produce pyoil stream 14 comprising pyrolysis oil. In embodiments of the invention, plastic stream 13 comprising thermally cracked and/or partially thermally cracked plastic is produced by processing the plastic in extruder 102. The extruder can be operated to provide an operating temperature of 300 to 500° C. and a residence time of 1 to 15 minutes. In embodiments of the invention, prior to processing in the extruder, the plastic may be processed in chlorine removal unit 106 to remove at least some chlorine from the plastic. In embodiments of the invention, the chlorine is removed from the plastic via density based separation or solvent system. Pyoil stream 14 at block 601 can include less than 30 ppmw chlorine.

According to embodiments of the invention, as shown in block 602, method 600 includes hydroprocessing, in hydroprocessing unit 104, pyoil stream 14 to produce hydroprocessed pyrolysis oil stream 20 comprising hydroprocessed pyoil. In embodiments of the invention, hydroprocessing at block 602 further removes chlorine from pyoil stream 14. Hydroprocessed pyrolysis oil stream 20 comprises less than 3 ppmw chlorine. In embodiments of the invention, hydroprocessed pyrolysis oil stream 20 meets the boiling range requirement for a steam cracker (e.g., steam cracker 115). Hydroprocessed pyrolysis oil stream 20 may comprise less than 20 wt. %, preferably less than 10 wt. % aromatics. Hydroprocessed pyrolysis oil stream 20 may comprise less than 3 ppm preferably less than 1 ppm chlorine. Hydroprocessed pyrolysis oil stream 20 may comprise less than 1 wt. % olefins. In embodiments of the invention, hydroprocessing unit 104 is operated to provide a reaction temperature of 350 to 500° C. and all ranges and values there between including ranges of 350 to 365° C., 365 to 380° C., 380 to 395° C., 395 to 410° C., 410 to 425° C., 425 to 440° C., 440 to 455° C., 455 to 470° C., 470 to 485° C., and 485 to 500° C. Hydroprocessing unit 104, at block 602, can be operated to provide an operating pressure of 20 to 100 barg and all ranges and values there between including ranges of 20 to 30 barg, 30 to 40 barg, 40 to 50 barg, 50 to 60 barg, 60 to 70 barg, 70 to 80 barg, 80 to 90 barg, and 90 to 100 barg. At block 602, hydroprocessing unit 104 can be operated to provide a weight hourly space velocity of 1 to 2 hr⁻¹. In embodiments of the invention, at block 602, hydroprocessing unit 104 can be operated to provide a hydrogen to hydrocarbon volumetric ratio of 400 to 2000 Nm³/m³ liquid feed.

According to embodiments of the invention, as shown in block 603, method 600 includes mixing hydroprocessed pyrolysis oil stream 20 with second fraction stream 19 comprising crude oil or a crude oil fraction to produce cracker feed stream 18. In embodiments of the invention, the crude oil can include light crude oil having a boiling range of less than 35 to 350° C. The crude oil fraction of second fraction stream 19 can include condensate and/or naphtha.

According to embodiments of the invention, as shown in block 604, method 600 includes cracking hydrocarbons of cracker feed stream 18 in a cracking unit (e.g., steam cracker 115) under reaction conditions sufficient to produce product stream 16 comprising one or more of C₂ to C₄ olefins, benzene, toluene, and xylene. In embodiments of the invention, the cracking unit is steam cracker 115. Steam cracker 115 is operated to provide an operating temperature of 700 to 900° C. and a residence time of 50 ms to 1 s. Steam cracker 115 is operated to provide a hydrocarbon to steam weight ratio of 0.2 to 1.

Although embodiments of the present invention have been described with reference to blocks of FIGS. 3A to 3D, it should be appreciated that operation of the present invention is not limited to the particular blocks and/or the particular order of the blocks illustrated in FIGS. 3A to 3D. Accordingly, embodiments of the invention may provide functionality as described herein using various blocks in a sequence different than that of FIGS. 3A to 3D.

The systems and processes described herein can also include various equipment that is not shown and is known to one of skill in the art of chemical processing. For example, some controllers, piping, computers, valves, pumps, heaters, thermocouples, pressure indicators, mixers, heat exchangers, and the like may not be shown.

As part of the disclosure of the present invention, specific examples are included below. The examples are for illustrative purposes only and are not intended to limit the invention. Those of ordinary skill in the art will readily recognize parameters that can be changed or modified to yield essentially the same results.

### Example 1

### Processing of Pyoil and a Crude Oil Fraction

A commercial plastic pyoil with the properties as shown in Table 1 entry 1 was mixed with a crude oil stream cut with boiling point distribution shown in Table 1 entry 2 to give a mixed stream with the boiling point distribution as in Table 1 entry 3. This material was fed into a hydroprocessing unit. The reaction was carried out in a tubular reactor of length 304.8 mm and internal diameter of 13.1 mm. The reactor was housed in a tubular furnace heated electrically. The unit had provisions to feed the liquid to the reactor through a High Performance Liquid Chromatography (HPLC) pump. Hydrogen gas was passed through a mass flow controller at 9.8 NLPH (normal liters per hour). The reactor was loaded with a combination of commercial catalysts capable of hydrocracking and hydrotreating in the sequence hydrotreating catalyst (4.84 g)-hydrocracking catalyst (9.8 g)-hydrotreating catalyst (4.84 g). The catalyst weights indicated are bone dry weights. All the catalysts were diluted with SiC (200-225 microns) in a proportion 60% SiC to 40% bone dry catalyst weight. The neutral alumina with 1 mm diameter was loaded in the reactor upstream and downstream of the catalyst bed. During the reaction, the reactor bed temperature was maintained at a temperature of 380° C., pressure at 60 barg, weight hourly space velocity of 1 h⁻¹ and hydrogen to hydrocarbon ratio of 400 Nm³ hydrogen/m³ hydrocarbon liquid. The reaction product was passed through a condenser and a gas liquid separator. The gas was analyzed using a refinery gas analyzer and the liquid was analyzed for its boiling point distribution using a SIM-DIS GC and for PIONA (paraffin, isoparaffin, olefin, naphthenes and aromatics) using a 2D GC. The results of the analysis are in Table 1. The boiling point distribution of the feed to the hydroprocessing unit has 27 wt. % material boiling below 150° C. (Final boiling point at 381° C.) while the liquid product had 80% boiling point below 150° C., a significant upgrade to lighter products with the final boiling point at 309° C. (FIG. 2A). This liquid product is a light feed to steam cracker. Also, the 2D GC analysis of the product and feed reveals that the aromatics had been reduced from 16.7 wt. % in feed to 10.1 wt. % in the liquid product. Moreover, the total concentration of di- and tri-aromatics as well as naphthenic aromatics have dropped from 6% in the feed to 0.3% in the product. The branched naphthenes and dinaphthenes were reduced from 34% to 22%. These results indicate significant ring opening activity and conversion to saturates. There was a net increase in isoparaffin content of liquid product. In addition, about 10% gas was produced which can be further cracked to ethylene in a steam cracker.

TABLE 1
Boiling Point distribution in ° C. vs Mass
% of feed and product from the hydroprocessing unit

| | | Entry-2 | Entry-3 | |
|---|---|---|---|---|
| | | 350 minus | Feed 90/10 | Entry-4 |
| | Entry-1 | cut of | 350 minus | (90:10) |
| | Commercial | WTB | cut of WTB + | Hydroprocessing |
| Mass % | Pyoil | crude oil | pyoil | Unit Product |
| 0 | 86.4 | 70.2 | 70.2 | 69.0 |
| 5 | 124.8 | 94.4 | 94.4 | 69.0 |
| 10 | 131.8 | 94.4 | 107.6 | 69.0 |
| 15 | 150.8 | 107.6 | 107.6 | 69.0 |
| 20 | 174.4 | 124.8 | 131.8 | 86.4 |
| 25 | 191.8 | 138.2 | 144.6 | 94.4 |
| 30 | 203.6 | 153.4 | 160.6 | 94.4 |
| 35 | 217.8 | 170.2 | 176.6 | 94.4 |
| 40 | 235.4 | 181.6 | 188.8 | 94.4 |
| 45 | 249.6 | 198 | 202.2 | 94.4 |
| 50 | 260.6 | 210.8 | 218.0 | 107.6 |
| 55 | 273.4 | 222.2 | 228.0 | 107.6 |
| 60 | 287.6 | 235.4 | 240.6 | 107.6 |
| 65 | 300.2 | 247.6 | 254.4 | 124.8 |
| 70 | 309.2 | 259 | 267.4 | 124.8 |
| 75 | 319.2 | 273.4 | 281.4 | 138.2 |
| 80 | 332.6 | 290.4 | 298.0 | 150.8 |
| 85 | 345.8 | 305.4 | 311.6 | 163.0 |
| 90 | 358.4 | 319.2 | 325.2 | 182.6 |
| 95 | 375.6 | 333.2 | 341.0 | 218.6 |
| 99 | 406.8 | 350.4 | 366.4 | 286.4 |
| 100 | 468.8 | 355.6 | 381.4 | 308.6 |

TABLE 2
PIONA analysis of the product and feed of Example 1

| | Feed 90/10 350° | |
|---|---|---|
| | C. minus cut of | Hydroprocessing |
| Components | WTB + pyoil | Unit Product |
| Normal Paraffin | 20.4 | 16.5 |
| Isoparaffin | 21.5 | 40.7 |
| Naphthenes | 7.4 | 10.8 |
| Branched Naphthenes | 26.1 | 20.7 |
| Dinaphthenes | 7.9 | 1.2 |
| Monoaromatics | 10.7 | 9.8 |
| Naphthenic aromatics | 2.8 | 0.3 |
| Diaromatics | 2.1 | 0.02 |
| Naphthenic Diaromatics | 0.1 | 0 |
| Triaromatics | 1 | 0 |

TABLE 3
Crackability of (less than) 350° C. crude oil cut (based on
published information of light naphtha, heavy naphtha and gas
oil cracking) and product from hydroprocessing unit in steam cracker

| | | Typical Yields | |
|---|---|---|---|
| | | on Feeding | |
| | Typical | Hydroprocessing | |
| | Yields of | Reaction | |
| | 350 minus | Product to | |
| Products | feed to SC | Steam Cracker | Delta |
| Ethylene | 31 | 37 | +6 |
| Fuel oil | 11 | 3 | +8 |
| Methane | 14 | 18 | +4 |
| Hydrogen | 1 | 1 | Neutral |

As can be seen in Table 3, there is potential for 6% higher ethylene yield, 8% lower fuel oil make when the hydroprocessing unit product is processed in steam cracker as compared to processing (less than) 350° C. cut in steam cracker. This data substantiates the benefits of backward integration of steam cracker to hydroprocessing unit.

### Example 2

### Boiling Point Distribution Comparison Between a Crude Oil Cut and Hydroprocessing Product

The boiling point distribution comparison of a boiling cut with a from West Texas Blend crude oil was compared with hydroprocessing unit product from processing whole crude oil. The boiling point distributions appear to be similar. This shows that a heavier crude oil can be processed in hydroprocessing unit to reduce it to lower than 350° C. boiling cut, which is similar to condensate range material processed in a steam cracker.

TABLE 4
Boiling point distribution comparison of a boiling cut with
a from West Texas Blend crude oil was compared with hydroprocessing
unit product from processing whole crude oil

| | Entry-1 | | Entry-3 |
|---|---|---|---|
| | WTB | Entry-2 | Hydroprocessing |
| | Whole | 350 minus | Product of WTB |
| Mass % | crude | cut of WTB | Whole Crude |
| 0 | 84.4 | 70.2 | 35 |
| 5 | 139.8 | 94.4 | 84.4 |
| 10 | 162.8 | 94.4 | 102 |
| 15 | 183.2 | 107.6 | 112.2 |
| 20 | 204.2 | 124.8 | 122.4 |
| 25 | 222.4 | 138.2 | 136 |
| 30 | 240.2 | 153.4 | 151 |
| 35 | 258.8 | 170.2 | 162.8 |
| 40 | 278 | 181.6 | 176.4 |
| 45 | 298 | 198 | 192 |
| 50 | 315.4 | 210.8 | 202.8 |
| 55 | 338.4 | 222.2 | 217.6 |
| 60 | 362 | 235.4 | 229.8 |
| 65 | 390.6 | 247.6 | 245.4 |
| 70 | 424.6 | 259 | 263.4 |
| 75 | 466.4 | 273.4 | 284.2 |
| 80 | 499 | 290.4 | 286.4 |
| 85 | 526.8 | 305.4 | 287.4 |
| 90 | 560.2 | 319.2 | 305.8 |
| 95 | 611.2 | 333.2 | 331.8 |
| 99 | 687.4 | 350.4 | 377.4 |
| 100 | 705.6 | 355.6 | 390.2 |

### Example 3

### Boiling Point Distribution Comparison Between a Crude Oil Cut and Hydroprocessing Product

100% commercial pyoil with boiling point distribution as in Table 1 entry 1 was processed in the hydroprocessing unit as in Example 1 under the same conditions except that the temperature severity was varied from 380° C. to 400° C. The gas was analyzed using a refinery gas analyzer and the liquid was analyzed for its boiling point distribution using a simulated distillation gas chromatography (SIM-DIS GC) and for PIONA (paraffin, isoparaffin, olefin, naphthenes and aromatics) using a 2D GC. The results of the analysis are in Table 3. The boiling point distribution of the feed to the hydroprocessing unit has 15 wt. % material boiling point below 150° C. (Final boiling point at 468° C.) while the liquid product had 30% boiling below 150° C. The product obtained at 400° C. reaction temperature has a higher percentage of products boiling below 150° C. Although the upgradation is less compared to the product obtained in Example 1 where the pyoil was blended with crude oil stream cut, the majority of the product (>98%) boils below the steam cracker feed requirement of <350° C. Also, the 2D GC analysis of the product and feed reveals that the aromatics had been reduced from 14 wt. % in feed to 8.6 wt. % in the liquid product at 380 and 9.2 wt. % at 400° C. This is a clear indication that aromatics increase with temperature severity. So an effort to reduce the boiling point well below 350° C. by applying temperature severity is possible but would come at the expense of making more aromatics and hence a greater tendency to form coke. Hence, a balance needs to be struck between maintaining the aromatics below 10% and boiling point of the majority of the product below 350° C. by tweaking the temperature severity. Moreover, the total concentration of di- and tri-aromatics as well as naphthenic aromatics have dropped from 5.7% in the feed to 1.4% in the product. The branched naphthenes and dinaphthenes have reduced from 42% to 14.9%. These results indicate significant ring opening activity and conversion to saturates. There was a net increase in paraffin content of liquid product.

TABLE 5
Boiling Point distribution in ° C. vs Mass
% of commercial pyoil as feed and product from
the SURF unit at different temperature severity

| | | Entry-2 | Entry-3 |
|---|---|---|---|
| | Entry-1 | Product from | Product from |
| | Commercial | Hydroprocessing | Hydroprocessing |
| Mass % | Pyoil | at 380° C. | at 400° C. |
| 0 | 86.4 | 69 | 69 |
| 5 | 124.8 | 94.4 | 69 |
| 10 | 131.8 | 107.6 | 94.4 |
| 15 | 150.8 | 107.6 | 94.4 |
| 20 | 174.4 | 124.8 | 107.6 |
| 25 | 191.8 | 138.2 | 107.6 |
| 30 | 203.6 | 153.4 | 107.6 |
| 35 | 217.8 | 175.4 | 124.8 |
| 40 | 235.4 | 187.8 | 138.2 |
| 45 | 249.6 | 200.8 | 153.4 |
| 50 | 260.6 | 219.2 | 172.6 |
| 55 | 273.4 | 236.6 | 183.6 |
| 60 | 287.6 | 250.2 | 197.4 |
| 65 | 300.2 | 265 | 215.6 |
| 70 | 309.2 | 278.6 | 233.6 |
| 75 | 319.2 | 293.2 | 252 |
| 80 | 332.6 | 308 | 269.8 |
| 85 | 345.8 | 323.6 | 286.6 |
| 90 | 358.4 | 342.4 | 305.6 |
| 95 | 375.6 | 360.2 | 329.6 |
| 99 | 406.8 | 386.6 | 365.8 |
| 100 | 468.8 | 393.2 | 376.2 |

TABLE 6
PIONA analysis of the products and feed of Example 3

| | 100% | | |
|---|---|---|---|
| | Commercial | Product at | Product at |
| Components | pyoil | 380° C. | 400° C. |
| Normal Paraffin | 31 | 44.8 | 34.5 |
| Isoparaffin | 9.7 | 25.4 | 36.2 |
| Naphthenes | 3.3 | 4.8 | 5.2 |
| Branched Naphthenes | 36 | 14.2 | 13.6 |
| DiNaphthenes | 6 | 2.2 | 1.3 |
| Monoaromatics | 8.3 | 6.7 | 7.8 |
| Naphthenic aromatics | 2.7 | 1.7 | 1.3 |
| Diaromatics | 1.5 | 0.1 | 0.1 |
| Naphthenic Diaromatics | 0.5 | 0.04 | 0 |
| Triaromatics | 1 | 0.06 | 0 |

It is observed that lower processing temperature results in more n-paraffins.

### Example 4

A portion of the West Texas Blend crude oil boiling in the light vacuum gas oil (VGO) range of 370° C. to 415° C. was fed to an in situ fluidized bed pyrolysis lab reactor using N₂ as a carrier gas at a flow rate of 175 Ncc/min. The in situ fluidized bed reactor has a length of 783 mm and an inner diameter of 15 mm and housed in a split zone 3 zone tubular furnace with independent temperature control for each zone. The size of each zone was 9.3 inches (236.2 mm). The overall heated length of the reactor placed inside the furnace was 591 mm. The reactor wall temperature was measured at the center of each zone and was used to control the heating of each furnace zone. The reactor had a conical bottom and the reactor bed temperature was measured using a thermocouple housed inside a thermowell and placed inside the reactor at the top of the conical bottom. Also, the reactor wall temperature was measured at the conical bottom to ensure that the bottom of the reactor was hot. The reactor bottom was placed at the middle of the furnace bottom zone for minimizing the effect of furnace end cap heat losses and maintaining the reactor bottom wall temperature within a difference of 20° C. of the internal bed temperature measured. This experiment was carried out in high severity pyrolysis mode i.e. in the absence of hydrogen in the carrier gas and at high temperature. The experimental conditions are as mentioned in Table 7. As soon as feed was introduced into the reactor, the reactor inside temperature dropped quite rapidly due to endothermic cracking and vaporization and also comes back to the reactor bed temperature existing before feed introduction within a minute. About 60% of the product formed within the first 1 minute after feed introduction and the temperature severity during this 1 minute governs the overall product yield and selectivity. The 1 minute time averaged reaction bed temperature was calculated based on the experimental bed temperature within the first 1 minute and is reported in Table 7. Apart from the Cat/Oil ratio, the 1 minute average bed temperature is a parameter indicating reaction severity. The yields from experiment 4 are mentioned in Table 7. The Ethylene to Propylene ratio (E/P) was between 0.9 to 1 wt. %. The light gas yield per unit coke is around 9 to 10. In pilot or commercial reactors where the gas flow rates (improved mixing and catalyst contact with feed) and heat transfer rates are much higher it is very much possible to obtain higher yields of high value chemicals under similar conditions.

TABLE 7
High severity FCC of West Texas Blend
crude oil boiling in light VGO range

| Run # | | |
|---|---|---|
| Process | High Severity | High Severity |
| | pyrolysis | Pyrolysis |
| Feed type | Liquid | Liquid |
| Feed name | West Texas | West Texas |
| | Blend crude oil | Blend crude oil |
| | 370 to 415° C. | 370 to 415 ° C. |
| | Boiling cut | Boiling cut |
| Fluidization gas composition | 100% N₂ | 100% N₂ |
| (mole %) | | |
| C/F ratio | 6.2 | 5.8 |
| Reaction temperature at start, ° C. | 700 | 700 |
| 1 min average reactor bed | 688 | 687 |
| temperature, ° C. | | |
| Bone Dry catalyst fed | 9.4 | 9.1 |
| Feed weight transferred | 1.53 | 1.57 |
| Yields, wt. % on product basis | | |
| C₆+ | 18.2 | 18.1 |
| H₂ | 0.3 | 0.3 |
| Methane | 7.5 | 7.0 |
| Ethane | 4.3 | 4.3 |
| Ethylene | 12.1 | 11.8 |
| Carbon dioxide | 0.6 | 0.0 |
| Propane | 2.0 | 2.1 |
| Propylene | 12.1 | 13.0 |
| i-butane | 1.6 | 1.8 |
| n-butane | 0.5 | 0.6 |
| t-2-butene | 2.0 | 2.3 |
| 1-butene | 1.4 | 1.5 |
| i-butylene | 2.8 | 3.1 |
| c-2-butene | 1.5 | 1.7 |
| i-pentane | 0.8 | 0.9 |
| n-pentane | 0.0 | 0.0 |
| 1,3-butadiene | 1.1 | 0.7 |
| 2-methyl 1-butene | 1.7 | 1.9 |
| t-2-pentene | 0.0 | 0.0 |
| 2-methyl 2-butene | 1.2 | 1.4 |
| 1-pentene | 0.4 | 0.5 |
| 3-methyl 1-butene | 0.0 | 0.0 |
| c-2-pentene | 0.0 | 0.0 |
| Nitrogen | | |
| Oxygen | | |
| Unknown | 1.8 | 1.4 |
| Carbon monoxide | 0.0 | 0.0 |
| Gasoline | 14.4 | 8.5 |
| Diesel | 5.2 | 8.5 |
| Heavies | 2.8 | 5.4 |
| Coke | 3.7 | 3.3 |
| Naphtha carried with gas | 24.2 | 24.2 |
| Corrected Yields of gasoline after | 38.5 | 32.7 |
| dropping C₅ and C₆ from gas | | |
| % of i-C₄/total C₄ | 25.6 | 26.5 |
| % of olefins/total gases | 66.2 | 67.9 |
| % of olefins/% saturates | 3.9 | 3.9 |
| % H₂, C₁, C₄ yield | 49.8 | 50.1 |
| % liquid yield | 46.5 | 46.5 |
| % coke yield | 3.7 | 3.3 |
| C₄ = wt. % | 8.7 | 9.2 |
| C₃ = wt. % | 12.1 | 13.0 |
| C₂ = wt. % | 12.1 | 11.8 |
| Total light gas olefins | 33.0 | 34.0 |
| Light gas olefins per unit coke | 9.0 | 10.2 |
| C₄ sat | 2.1 | 2.3 |
| C₃ sat | 2.0 | 2.1 |
| C₂ sat | 4.3 | 4.3 |
| Light gas saturates | 8.4 | 8.7 |
| Ethylene/propylene ratio | 1.0 | 0.9 |
| C₂ = C₂ sat | 2.8 | 2.8 |
| C₃ = C₃ sat | 6.2 | 6.1 |
| C₄ = C₄ sat | 4.1 | 3.9 |
| Light gas olefins/light gas Saturates | 3.9 | 3.9 |
| Hydrogen Transfer Index (HTI) | 0.3 | 0.3 |
| Isomerization coefficient | 0.2 | 0.2 |

### Example 5

Experiments were carried out with portion of West Texas Blend crude oil boiling in the light VGO range of 370° C. to 415° C. as in Experiment 4 except that the unit was operated under hydropyrolysis mode with the fluidization gas composition of N₂/H₂ as 90/10 mole %. Results are tabulated in Table 8. By operating under hydropyrolysis mode, the light gas olefins yield has increased, coke has reduced, thereby the light gas olefins/unit coke has increased. In addition to this, heavies formation has reduced. This is a clear indication that HVC yield can be increased by altering the fluidizing gas composition. Other handles which can be exploited for improved yields in commercial plants include temperature profiles, residence time changes, reactor section designs and catalyst recipe are covered in U.S. Patent Application No. 63/109,507 and are incorporated by reference herein.

TABLE 8
Hydropyrolysis of West Texas Blend crude oil boiling
in light VGO range in a High severity FCC unit

| Process | Hydropyrolysis | Hydropyrolysis | Hydropyrolysis |
|---|---|---|---|
| Feed type | Liquid | Liquid | Liquid |
| Feed name | West Texas | West Texas | West Texas |
| | Blend crude oil | Blend crude oil | Blend crude oil |
| | 370 to 415° C. | 370 to 415° C. | 370 to 415° C. |
| | Boiling cut | Boiling cut | Boiling cut |
| Fluidization gas composition | 90/10 N₂/H₂ | 90/10 N₂/H₂ | 90/10 N₂/H₂ |
| (mole %) | | | |
| C/F ratio | 6.0 | 6.1 | 5.930838833 |
| Reaction temperature at start, | 700 | 700 | 700 |
| ° C. | | | |
| 1 min average reactor bed | 686 | 685 | 688 |
| temperature ° C. | | | |
| Bone Dry catalyst fed | 350.0 | 9.1 | 9.0 |
| Feed weight transferred | 1.51 | 1.50 | 1.53 |
| Yields, wt. % on product basis | | | |
| C₆+ | 18.5 | 17.7 | 19.7 |
| H₂ | 0.0 | 0.0 | 0.0 |
| Methane | 7.9 | 7.7 | 11.1 |
| Ethane | 4.6 | 4.4 | 5.6 |
| Ethylene | 12.4 | 12.7 | 14.9 |
| Carbon dioxide | 1.0 | 0.9 | 1.1 |
| Propane | 2.3 | 1.8 | 2.2 |
| Propylene | 14.0 | 14.6 | 12.8 |
| i-butane | 1.8 | 1.3 | 1.8 |
| n-butane | 0.6 | 0.5 | 0.6 |
| t-2-butene | 2.3 | 2.1 | 2.0 |
| 1-butene | 1.6 | 1.7 | 1.5 |
| i-butylene | 3.3 | 3.1 | 2.8 |
| c-2-butene | 1.7 | 1.5 | 1.5 |
| i-pentane | 1.0 | 0.8 | 0.9 |
| n-pentane | 0.0 | 0.0 | 0.0 |
| 1,3-butadiene | 0.9 | 1.3 | 1.3 |
| 2-methyl 1-butene | 0.0 | 0.0 | 0.0 |
| t-2-pentene | 0.7 | 0.6 | 0.6 |
| 2-methyl 2-butene | 1.4 | 1.1 | 1.2 |
| 1-pentene | 0.0 | 0.0 | 0.0 |
| 3-methyl 1-butene | 0.0 | 1.2 | 0.0 |
| c-2-pentene | 0.6 | 0.0 | 0.0 |
| Nitrogen | | | |
| Oxygen | | | |
| Unknown | 0.0 | 1.3 | 1.5 |
| Carbon monoxide | 0.0 | 0.0 | 1.1 |
| Gasoline | 12.4 | 16.1 | 8.3 |
| Diesel | 5.7 | 4.4 | 3.9 |
| Heavies | 2.8 | 1.2 | 1.5 |
| Coke | 2.5 | 2.2 | 2.2 |
| Naphtha carried with gas | 22.2 | 22.7 | 24.0 |
| Corrected Yields of gasoline | 34.6 | 38.8 | 32.3 |
| after dropping C₅ and C₆ from | | | |
| gas | | | |
| % of i-C₄/total C₄ | 26.8 | 26.8 | 24.2 |
| % of olefins/total gases | 66.5 | 69.0 | 62.2 |
| % of olefins/% saturates | 3.9 | 4.6 | 3.6 |
| % H₂, C₁, C₄ yield | 54.3 | 53.5 | 59.1 |
| % Liquid yield | 43.2 | 44.3 | 37.6 |
| % Coke yield | 2.5 | 2.2 | 2.2 |
| C₄ = wt. % | 9.8 | 9.6 | 9.1 |
| C₃ = wt. % | 14.0 | 14.6 | 12.8 |
| C₂ = wt. % | 12.4 | 12.7 | 14.9 |
| Total light gas olefins | 36.1 | 36.9 | 36.7 |
| Light gas olefins per unit coke | 14.6 | 17.1 | 16.4 |
| C₄ sat | 2.4 | 1.8 | 2.3 |
| C₃ sat | 2.3 | 1.8 | 2.2 |
| C₂ sat | 4.6 | 4.4 | 5.6 |
| Light gas saturates | 9.3 | 8.0 | 10.1 |
| Ethylene/Propylene ratio | 0.9 | 0.9 | 1.2 |
| C₂ = C₂ sat | 2.7 | 2.9 | 2.7 |
| C₃ = C₃ sat | 6.1 | 8.0 | 5.9 |
| C₄ = C₄ sat | 4.0 | 5.3 | 3.9 |
| Light gas olefins/light gas | 3.9 | 4.6 | 3.6 |
| saturates | | | |
| Hydrogen Transfer Index (HTI) | 0.3 | 0.2 | 0.4 |
| Isomerization coefficient | 0.2 | 0.3 | 0.2 |

In the context of the present invention, at least the following 20 embodiments are described. Embodiment 1 is a method of processing hydrocarbons. The method includes hydroprocessing, in a hydroprocessing unit, a hydrocarbon stream containing: (1) a pyrolysis oil obtained from a plastic and (2) at least a fraction from a crude oil distillation unit under reaction conditions sufficient to produce a cracker feed stream. The method further includes cracking, in a fluid catalytic cracking unit, hydrocarbons of the cracker feed stream in the presence of a catalyst under reaction conditions sufficient to produce a product stream containing one or more of C₂ olefins, C₃ olefins, C₄ olefins, benzene, toluene, and xylene.

Embodiment 2 is a method of processing hydrocarbons. The method includes hydroprocessing, in a hydroprocessing unit, a fraction from a crude oil distillation unit under reaction conditions sufficient to produce a hydroprocessed liquid stream. The method further includes mixing the hydroprocessed liquid stream and a pyrolysis oil obtained from a plastic to form a cracker feed stream. The method still further includes cracking, in a fluid catalytic cracking unit, hydrocarbons of the cracker feed stream in the presence of a catalyst under reaction conditions sufficient to produce a product stream containing one or more of C₂ olefins, C₃ olefins, C₄ olefins, benzene, toluene, and xylene. Embodiment 3 is the method of any of embodiments 1 and 2, wherein the fraction from the crude oil distillation unit contains atmospheric residue and vacuum gas oil. Embodiment 4 is the method of any of embodiments 1 to 3, wherein the hydroprocessing includes low pressure hydrocracking/hydrotreating. Embodiment 5 is the method of any of embodiments 1 to 4, wherein the low pressure hydrocracking hydrotreating is conducted in a hydroprocessing unit operated under a pressure of less than 100 barg. Embodiment 6 is the method of any of embodiments 1 to 5, wherein the hydroprocessing unit is configured to be operated in hydrocracking mode and/or hydrotreating mode. Embodiment 7 is the method of any of embodiments 1 to 6, wherein the catalyst used in the hydroprocessing step includes a hydrotreating catalyst and/or a hydrocracking catalyst. Embodiment 8 is the method of any of embodiments 1 to 7, wherein the hydrotreating catalyst includes CoMo, NiMo, CoNiMo, NiW, NiWMo, or combination on alumina or silica or aluminosilicates, and the hydrocracking catalyst includes CoMo, NiMo, CoNiMo, NiW, NiWMo or combinations on alumina or silica or aluminosilicates or zeolites such as X-type zeolites, Y-type or USY-type zeolites, mordenite, faujasite, nano-crystalline zeolites, MCM mesoporous materials, SBA-15, silico-alumino phosphate, gallophosphate, titanophosphate, ZSM-5, ZSM-11, ferrierite, heulandite, zeolite-A, erionite, and chabazite, or combinations thereof. Embodiment 9 is the method of any of embodiments 1 to 8, wherein the pyrolysis oil is produced by the steps of processing the plastic in an extruder to produce an extruder effluent, and processing the extruder effluent in a catalytic cracking unit under reaction conditions sufficient to produce the pyrolysis oil. Embodiment 10 is the method of any of embodiments 1 to 9, wherein the steps of producing pyrolysis oil further include removing, prior to processing in the extruder, at least some chlorine from the plastic. Embodiment 11 is the method of any of embodiments 1 to 10, wherein the removing step is conducted via density based separation and solvent system. Embodiment 12 is the method of any of embodiments 1 to 11, wherein the fluid catalytic cracking unit is operated under hydropyrolysis conditions.

Embodiment 13 is a method of processing hydrocarbons. The method includes processing a plastic stream containing a thermally cracked and/or partially thermally cracked plastic in a fixed bed fluid catalytic cracking unit under reaction conditions sufficient to crack at least some hydrocarbons of the thermally cracked and/or partially thermally cracked plastic, remove at least some of chlorine from the thermally cracked and/or partially thermally cracked plastic, and produce a pyrolysis oil stream containing pyrolysis oil. The method further includes hydroprocessing, in a hydroprocessing unit, the pyrolysis oil stream and crude oil or a crude oil fraction to produce a cracker feed stream. The method still further includes cracking hydrocarbons of the cracker feed stream in a cracking unit under reaction conditions sufficient to produce a product stream containing one or more C₂ olefins, C₃ olefins, C₄ olefins, benzene, toluene, and xylene.

Embodiment 14 is a method of processing hydrocarbons. The method includes processing a plastic stream containing a thermally cracked and/or partially thermally cracked plastic in a fixed bed fluid catalytic cracking unit under reaction conditions sufficient to crack at least some hydrocarbons of the thermally cracked and/or partially thermally cracked plastic, remove at least some of chlorine from the thermally cracked and/or partially thermally cracked plastic, and produce a pyrolysis oil stream containing pyrolysis oil. The method further includes hydroprocessing, in a hydroprocessing unit, the pyrolysis oil stream to produce a hydroprocessed pyrolysis oil stream. The method still further includes mixing the hydroprocessed pyrolysis oil stream with crude oil or a crude oil fraction to produce a cracker feed stream. The method also includes cracking hydrocarbons of the cracker feed stream in a cracking unit under reaction conditions sufficient to produce a product stream containing one or more of C₂ olefins, C₃ olefins, C₄ olefins, benzene, toluene, and xylene. Embodiment 15 is the method of any of embodiments 13 and 14, wherein the hydroprocessing includes hydrocracking and/or hydrotreating. Embodiment 16 is the method of any of embodiments 13 to 15, wherein the hydrotreating is configured to produce a hydrocarbon stream having a chlorine concentration of less than 3 ppmw. Embodiment 17 is the method of any of embodiments 13 to 16, wherein the catalyst includes: (a) a hydrotreating catalyst including CoMo, NiMo, CoNiMo, NiW, NiWMo, or combination on alumina or silica or aluminosilicates, and/or (b) a hydrocracking catalyst including CoMo, NiMo, CoNiMo, NiW, NiWMo or combinations on alumina or silica or aluminosilicates or zeolites such as X-type zeolites, Y-type or USY-type zeolites, mordenite, faujasite, nano-crystalline zeolites, MCM mesoporous materials, SBA-15, silico-alumino phosphate, gallophosphate, titanophosphate, ZSM-5, ZSM-11, ferrierite, heulandite, zeolite-A, erionite, and chabazite, or combinations thereof. Embodiment 18 is the method of any of embodiments 13 to 17, wherein the crude oil distillation unit includes condensate and/or naphtha. Embodiment 19 is the method of any of embodiments 13 to 18, wherein the pyrolysis oil is produced by steps of processing a plastic in an extruder to form a partially cracked plastic fraction, and catalytically cracking partially cracked plastic fraction in a fixed bed reactor containing a ZSM-5 or metal loaded ZSM-5 catalyst under reaction conditions sufficient to produce the pyrolysis oil. Embodiment 20 is the method of any of embodiments 13 to 19, wherein the steps for producing the pyrolysis oil further include removing, prior to processing in the extruder, at least some chlorine from the plastic.

All embodiments described above and herein can be combined in any manner unless expressly excluded.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of processing hydrocarbons, the method comprising:
hydroprocessing, in a hydroprocessing unit, a hydrocarbon stream comprising:
(1) a pyrolysis oil obtained from a plastic and (2) at least a fraction from a crude oil distillation unit under reaction conditions sufficient to produce a cracker feed stream; and
cracking, in a fluid catalytic cracking unit, hydrocarbons of the cracker feed stream in the presence of a catalyst under reaction conditions sufficient to produce a product stream comprising one or more of C₂ olefins, C₃ olefins, C₄ olefins, benzene, toluene, and xylene.

2. A method of processing hydrocarbons, the method comprising:
hydroprocessing, in a hydroprocessing unit, a fraction from a crude oil distillation unit under reaction conditions sufficient to produce a hydroprocessed liquid stream;
mixing the hydroprocessed liquid stream and a pyrolysis oil obtained from a plastic to form a cracker feed stream;
cracking, in a fluid catalytic cracking unit, hydrocarbons of the cracker feed stream in the presence of a catalyst under reaction conditions sufficient to produce a product stream comprising one or more of C₂ olefins, C₃ olefins, C₄ olefins, benzene, toluene, and xylene.

3. The method of claim 1, wherein the fraction from the crude oil distillation unit comprises atmospheric residue and vacuum gas oil.

4. The method of claim 1, wherein the hydroprocessing comprises low pressure hydrocracking/hydrotreating.

5. The method of claim 4, wherein the low pressure hydrocracking hydrotreating is conducted in a hydroprocessing unit operated under a pressure of less than 100 barg.

6. The method of claim 5, wherein the hydroprocessing unit is configured to be operated in hydrocracking mode and/or hydrotreating mode.

7. The method of claim 1, wherein the catalyst used in the hydroprocessing step includes a hydrotreating catalyst and/or a hydrocracking catalyst.

8. The method of claim 7, wherein the hydrotreating catalyst comprises CoMo, NiMo, CoNiMo, NiW, NiWMo, or combination on alumina or silica or aluminosilicates, and the hydrocracking catalyst comprises CoMo, NiMo, CoNiMo, NiW, NiWMo or combinations on alumina or silica or aluminosilicates or zeolites such as X-type zeolites, Y-type or USY-type zeolites, mordenite, faujasite, nano-crystalline zeolites, MCM mesoporous materials, SBA-15, silico-alumino phosphate, gallophosphate, titanophosphate, ZSM-5, ZSM-11, ferrierite, heulandite, zeolite-A, erionite, and chabazite, or combinations thereof.

9. The method of claim 1, wherein the pyrolysis oil is produced by the steps of:
processing the plastic in an extruder to produce an extruder effluent; and
processing the extruder effluent in a catalytic cracking unit under reaction conditions sufficient to produce the pyrolysis oil.

10. The method of claim 9, wherein the steps of producing pyrolysis oil further comprise:
removing, prior to processing in the extruder, at least some chlorine from the plastic.

11. The method of claim 10, wherein the removing step is conducted via density based separation and solvent system.

12. The method of claim 1, wherein the fluid catalytic cracking unit is operated under hydropyrolysis conditions.

13. A method of processing hydrocarbons, the method comprising:
processing a plastic stream comprising a thermally cracked and/or partially thermally cracked plastic in a fixed bed fluid catalytic cracking unit under reaction conditions sufficient to crack at least some hydrocarbons of the thermally cracked and/or partially thermally cracked plastic, remove at least some of chlorine from the thermally cracked and/or partially thermally cracked plastic, and produce a pyrolysis oil stream comprising pyrolysis oil;
hydroprocessing, in a hydroprocessing unit, the pyrolysis oil stream and crude oil or a crude oil fraction to produce a cracker feed stream;
cracking hydrocarbons of the cracker feed stream in a cracking unit under reaction conditions sufficient to produce a product stream comprising one or more C₂ olefins, C₃ olefins, C₄ olefins, benzene, toluene, and xylene.

14. A method of processing hydrocarbons, the method comprising:
processing a plastic stream comprising a thermally cracked and/or partially thermally cracked plastic in a fixed bed fluid catalytic cracking unit under reaction conditions sufficient to crack at least some hydrocarbons of the thermally cracked and/or partially thermally cracked plastic, remove at least some of chlorine from the thermally cracked and/or partially thermally cracked plastic, and produce a pyrolysis oil stream comprising pyrolysis oil;
hydroprocessing, in a hydroprocessing unit, the pyrolysis oil stream to produce a hydroprocessed pyrolysis oil stream;
mixing the hydroprocessed pyrolysis oil stream with crude oil or a crude oil fraction to produce a cracker feed stream;
cracking hydrocarbons of the cracker feed stream in a cracking unit under reaction conditions sufficient to produce a product stream comprising one or more of C₂ olefins, C₃ olefins, C₄ olefins, benzene, toluene, and xylene.

15. The method of claim 13, wherein the hydroprocessing comprises hydrocracking and/or hydrotreating.

16. The method of claim 15, wherein the hydrotreating is configured to produce a hydrocarbon stream having a chlorine concentration of less than 3 ppmw.

17. The method of claim 13, wherein the catalyst includes (a) a hydrotreating catalyst including CoMo, NiMo, CoNiMo, NiW, NiWMo, or combination on alumina or silica or aluminosilicates, and/or (b) a hydrocracking catalyst including CoMo, NiMo, CoNiMo, NiW, NiWMo or combinations on alumina or silica or aluminosilicates or zeolites such as X-type zeolites, Y-type or USY-type zeolites, mordenite, faujasite, nano-crystalline zeolites, MCM mesoporous materials, SBA-15, silico-alumino phosphate, gallophosphate, titanophosphate, ZSM-5, ZSM-11, ferrierite, heulandite, zeolite-A, erionite, and chabazite, or combinations thereof.

18. The method of claim 13, wherein the crude oil distillation unit includes condensate and/or naphtha.

19. The method of claim 13, wherein the pyrolysis oil is produced by steps of:
processing a plastic in an extruder to form a partially cracked plastic fraction; and
catalytically cracking partially cracked plastic fraction in a fixed bed reactor comprising a ZSM-5 or metal loaded ZSM-5 catalyst under reaction conditions sufficient to produce the pyrolysis oil.

20. The method of claim 19, wherein the steps for producing the pyrolysis oil further comprise:
removing, prior to processing in the extruder, at least some chlorine from the plastic.
